(19) United States
(12) Patent Application Publication
(10) Pub. No.: US 2024/0362258 A1
(43) Pub. Date: Oct. 31, 2024

(54) KEYWORD PHRASE ANALYSIS

(71) Applicant: Verana Health, Inc., San Francisco, CA (US)

(72) Inventors: Michael Chidi Mbagwu, San Jose, CA (US); Matthew Immanuel Cintron, Allentown, PA (US)

(21) Appl. No.: 18/140,569

(22) Filed: Apr. 27, 2023

(51) Int. Cl. G06F 16/33; G06F 16/338; G06F 16/36; G06F 40/279; G16H 10/60

(52) U.S. Cl. CPC G06F 16/3344; G06F 16/338; G06F 16/374; G06F 40/279; G16H 10/60

(57) ABSTRACT

Systems and methods presented herein provide means to process a target text, such as a medical record, in search of keyword(s). A set of medical records are accessed, where the set of medical records includes the target text. A set of target keywords are initialized. One or more natural language processing (NLP) models are used to expand the set of target keywords based on the initialized set of keywords. The one or more NLP models process the target text with the expanded set of target keywords to extract a set of found keywords. A report including the set of found keywords and at least one text excerpt from the target text is caused for display.

### BACKGROUND

Within natural language processing (NLP), there is a central problem of lacking knowledge of the contents of a target text prior to processing. In particular, this lack of knowledge makes setting initial target keywords for searching a target text difficult. Certain keywords or keyword phrases may have the same meaning, but have different spelling, grammar, or phrases. This gap in keyword coverage leaves space for errors in processing a target text. For example, searching for a keyword that is not present while a synonymous keyword is present can give a false negative. This problem is especially complex when dealing with large unstructured data, such as medical records (e.g., physician notes).

Searching a target text for a keyword conventionally makes use of hardcoded regular expressions (shortened as “regex” or “regexp”). Each regex defines a pattern of characters and can be used in a string search to find a match in an accompanying target text. Accordingly, searching for all keywords with different spelling, grammar, and/or phrases that have the same meaning requires building an ever-expanding list of regex expressions. This regex solution is time-intensive and prone to mistakes, especially false negatives. Existing solutions to this problem are not scalable or robust enough for use in searching large unstructured target texts.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a diagrammatic representation of an example system for keyword phrase analysis, according to some examples.

FIG. 2 is a diagrammatic representation of a keyword processing system, according to some examples.

FIG. 3A illustrates an example target text, according to some examples.

FIG. 3B illustrates an example target text, according to some examples.

FIG. 3C illustrates an example target text, according to some examples.

FIG. 4 illustrates several exemplary types of keyword expansions, according to some examples.

FIG. 5 illustrates an example report, according to some examples.

FIG. 6 illustrates an example report, according to some examples.

FIG. 7 is a flowchart of a method of target text processing, according to some examples.

FIG. 8 is a diagrammatic representation of a machine in the form of a computer system within which a set of instructions may be executed to cause the machine to perform any one or more of the methodologies discussed herein, according to some examples.

### DETAILED DESCRIPTION

Systems and methods described herein present a means for keyword phrase analysis to aide in searching large unstructured texts. Solutions presented herein make use of NLP and/or machine-learning (ML) to expand beyond conventional regex text searching. The term “keywords” as referred to herein represents a word or phrase which may be in a target text. Systems and methods herein provide a means for searching for keywords within target text.

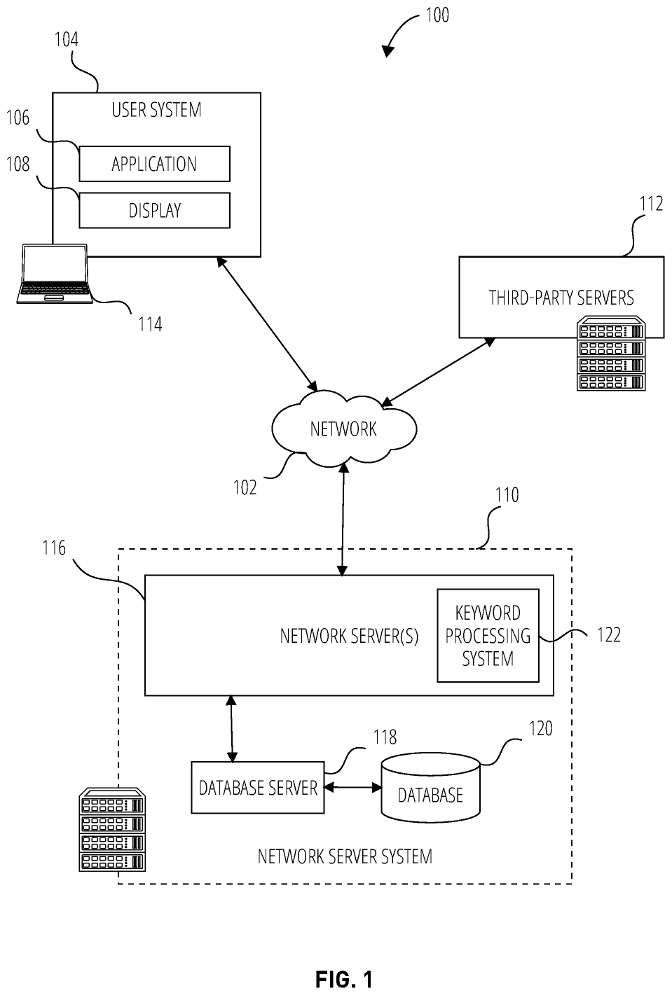

FIG. 1 is a block diagram showing an example system 100 for keyword phrase analysis. The system 100 includes at least one user system 104, each of which may host multiple applications, including an application 106. Each user system 104 is communicatively coupled, via a network 102 (e.g., the Internet), to a network server system 110 and third-party servers 112. Each user system 104 may include one or more user devices, such as a computer user device 114, that is communicatively connected to exchange data.

The application 106 provides processing and enables communication with the network 102. According to some examples, the application is a local client of a keyword processing system 122 and the application 106 is configured to provide the same processing and functionality as the keyword processing system 122.

The user system 104 includes a display 108 configured to display at least a user interface. According to some examples, the display 108 is integrated into the one or more user devices, such as the computer user device 114. The display 108 may be external to the user system 104 and be communicatively connected to the user system 104.

The user system 104 is connected with the network server system 110 via the network 102. The data exchanged between the one or more user systems 104 and the network server system 110 includes functions (e.g., commands to invoke functions) and payload data (e.g., text, audio, video, or other multimedia data).

The network server system 110 provides server-side functionality via the network 102 to the user system 104. While certain functions of the system 100 are described herein as being performed by either the user system 104 or the network server system 110, the location of certain functionality either within the user system 104 or the network server system 110 may be a design choice. For example, it may be technically preferable to initially deploy particular technology and functionality within the network server system 110 but to later migrate this technology and functionality to the application 106 where a user system 104 has sufficient processing capacity.

The network server system 110 supports various services and operations that are provided to the user system 104. Such operations include transmitting data to, receiving data from, and processing data generated by the user system 104 and/or third-party servers 112. This data may include one or more target texts, patient information, client device information, geolocation information, and other metadata. Data exchanges within the system 100 are invoked and controlled through functions available via user interfaces (UIs) of the application 106.

The network server system 110 includes one or more network servers 116 that provide processing functionality, making the functions of the network server system 110 accessible to the applications 106 of the user system 104 and/or the third-party server 112. The network servers 116 are communicatively coupled to a database server 118, facilitating access to a database 120 that stores data associated with interactions processed by the network servers 116. Similarly, the network servers 116 provides web-based interfaces and APIs, according to some examples. To this end, the network servers 116 process incoming network requests over the Hypertext Transfer Protocol (HTTP) and several other related protocols.

The network server system 110 receives and transmits interaction data (e.g., commands and data payloads) between the network servers 116 and the clients (for example, the application 106) and the third-party server 112. Specifically, the network server system 110 provides a set of interfaces (e.g., routines and protocols) that can be called or queried to invoke functionality of the network servers 116. The network server system 110 exposes various functions supported by the network servers 116, including account registration; login functionality; the sending of data, via the network servers 116, from a particular user system 104; the communication of files (e.g., images or other data) from the user system 104 to the network servers 116; the metadata of a collection of data (e.g., patient information); the storage or retrieval of a data (e.g., FAF images) from the database 120.

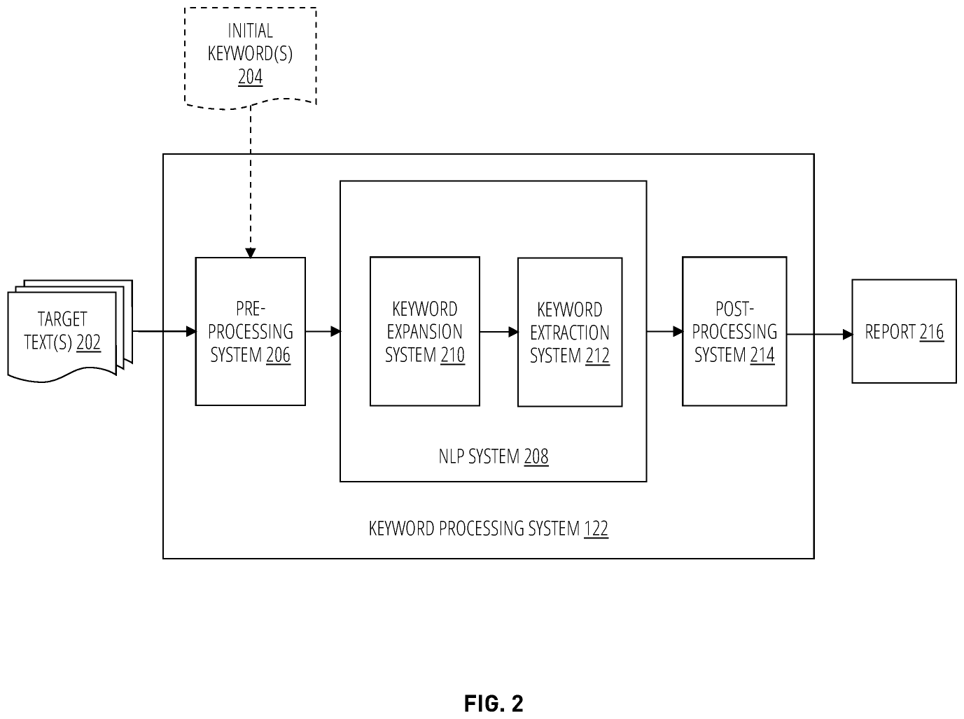

The network servers 116 host multiple systems and subsystems, including a keyword processing system 122 described below with reference to FIG. 2. The functionality of the keyword processing system 122 is described herein may be partially or fully performed an application 106 of an instance of the user system 104. Description of the keyword processing system 122 in reference to the network server system 110 is not meant to be limiting.

FIG. 2 is a diagrammatic representation of a keyword processing system 122, according to some examples. The keyword processing system 122 processes one or more target texts 202 to analyze the contents thereof to determine found keywords. The keyword processing system 122 receives at least one or more target texts 202 for processing. The target texts 202 contain at least text data. The target texts 202 may be raw text data or may be part of a document, for example, formats .txt, .docx, .pdf, .html, and the like. In instances where the target text 202 is a document, the target text 202 is associated with metadata about the document. The target texts 202 can be provided though user input, for example, through an input component of the user system 104 (e.g., computer user device 114).

According to some examples, the target texts 202 are medical texts, such as a note by a medical professional (e.g., a physician note), a report relating to a medical procedure, a report relating to imaging or other diagnostic tests, records of a patient's medical history and/or family medical history (e.g., patient intake form), documents or forms relating to insurance (e.g., health insurance, dental insurance, vision insurance, disability insurance, life insurance). Medical texts are generally large, unstructured texts, which makes them difficult to parse through for analysis. Additionally, keywords of interest in a medical context are often phrases rather than singular words, making keyword search through conventional regex string searches particularly cumbersome.

According to some examples, the keyword processing system 122 receives an initial set of keywords 204. The keyword processing system 122 initializes a set of keywords based on the received initial set of keywords 204. For example, the keyword processing system 122 receives the initial set of keywords 204 through a user input. The user input may be provided through an input component of the user system 104, such as the computer user device 114. The user input may specify a list of one or more keywords on which to base the processing of the target text 202. For example, the target text 202 is a report of a chest computed tomography (CT) scan and a user provides an initial set of keywords 204 that includes: “fluid,” “lesion,” “abnormal,” and “impression.”

In another example, the keyword processing system 122 receives the initial set of keywords 204 from a database (e.g., database 120). In such examples, the database may provide the initial set of keywords 204 based on context or user input. For example, an ophthalmologist's office using the keyword processing system 122 may make use of the same initial set of keywords 204 retrieved from the database relating to ophthalmological conditions. According to a related example, a user in the ophthalmologist's office may provide a user input to the keyword processing system 122 to select one option from a set of options for the initial set of keywords 204, for example, based on a patient's medical history.

According to other examples, the keyword processing system 122 does not receive an initial set of keywords 204 and initializes an empty initial set of keywords 204. Additionally, or alternatively, the keyword processing system 122 generates an initial set of keywords 204 based on names of medications. That is, the keyword processing system 122 generated an initial set of keywords 204 that includes a number of names of medications to initially search for in the target text 202.

The keyword processing system 122 includes a pre-processing system 206 that provides one or more pre-processing functions prior NLP processing. According to some examples, the pre-processing system 206 pre-processes the one or more target texts 202 and/or the initial set of keywords 204. Pre-processing a target text 202 and/or a keyword can include one or more of: removing formatting and excess spaces, removing punctuation and/or certain non-letter characters (e.g., <, >, #, /), removing non-English characters, removing stopwords (e.g., common words that do not contribute substantially to meaning, such as articles ‘a,’ ‘the,’ and the like), converting all text to lowercase, converting all text to uppercase, lemmatization, whitelisting, blacklisting, and other filters or reformatting that prepare for NLP processing. In examples where the keyword processing system 122 receives multiple target texts 202, the pre-processing system 206 combines the text data from each target text 202 into one text, according to some examples. In some examples, the pre-processing system 206 is optional and may be omitted.

The keyword processing system 122 includes an NLP system 208 that includes one or more NLP models. The NLP system 208 receives the pre-processed target texts 202 and, if applicable, the initial set of keywords 204 from the pre-processing system 206. The NLP system 208 provides one or more processing steps to search the target texts 202 to identify keywords. According to some examples, the search of the target texts 202 is based on the initial set of keywords 204. The NLP system 208 includes a keyword expansion system 210 and a keyword extraction system 212.

The keyword expansion system 210 expands the set of keywords. The keyword expansion system 210 employs the one or more NLP models of the NLP system 208 to identify additional target keywords. The keyword expansion system 210 provides an ability to expand a set of keywords beyond conventional regex. The keyword expansion system 210 receives at least the initialized set of keywords, according to some examples. In examples wherein the keyword processing system 122 receives an initial set of keywords 204, the keyword expansion system 210 expands the set of keywords based on the initial set of keywords 204. For example, given a particular keyword, the keyword expansion system 210 determines one or more additional keywords based on the particular keyword.

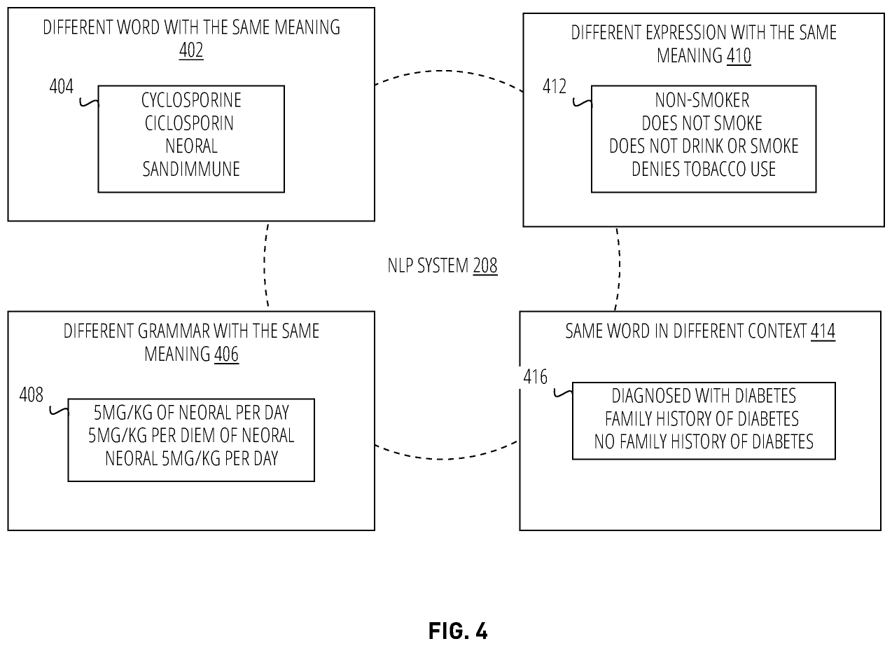

For example, the keyword expansion system 210 determines a first set of additional keywords that includes keywords that are synonyms of the given keyword (e.g., “Naproxen” and “N-Proxen”). The keyword expansion system 210 determines a second set of additional keywords that includes keywords with different grammar than the given keyword and synonymous meaning to the given keyword (e.g., “500 mg of Naproxen per day” and “per diem 500 mg Naproxen”). The keyword expansion system 210 determines a third set of additional keywords that includes keywords with different expressions than the given keyword and synonymous meaning to the given keyword (e.g., “non-smoker” and “does not smoke”). The keyword expansion system 210 determines a fourth set of additional keywords that includes keywords that comprise the given keyword in one or more different contexts (e.g., “Family history of cancer” and “No family history of cancer”). The expanded set of keywords includes any combination of the initial set of keywords 204, the first set of additional keywords, the second set of additional keywords, the third set of additional keywords, the fourth set of additional keywords. See FIG. 4 for further description of examples.

According to some examples, the one or more NLP models of the NLP system 208 that enable the keyword expansion system 210 employ bidirectional encoder representations from transformers (BERTs). For example, the one or more NLP models may use a sentence transforming BERT to compute grammatical and phrase-based transformations to expand the set of keywords. Additionally, or alternatively, the one or more NLP models may use a keyword-specific BERT to determine words or phrases most relevant in the target text 202. The keyword BERT can be employed in examples wherein the keyword processing system 122 does not receive an initial set of keywords 204 to extract a set of generalized keywords from the target text. Other BERTs or similar language transformation and processing frameworks may be employed by the keyword expansion system 210.

The keyword extraction system 212 extracts a set of found keywords from the target text 202. The keyword extraction system 212 employs one or more NLP models of the NLP system 208 to extract found keywords from the target text. The keyword extraction system 212 receives at least the target text 202, and the expanded set of keywords generated by the keyword expansion system 210, according to some examples.

The keyword extraction system 212 processes the target text 202 to find keywords from the expanded set of keywords in the target text 202. According to some examples, the one or more NLP models of the NLP system 208 that enable the keyword extraction system 212 employ BERTs. Sentence-based and/or keyword-based BERTs can be used to extract found keywords and determine relevant surrounding text. For example, for a given keyword, the keyword extraction system 212 determines whether the keyword was found and, if so, an excerpt of relevant text from the target text 202 that gives context to the found keyword. The keyword extraction system 212 may extract additional data about any found keywords, optionally in junction with the keyword expansion system 210.

It shall be appreciated by those of ordinary skill in the art that the keyword expansion system 210 and the keyword extraction system 212 are not necessarily two distinct systems and may share functions and processing between one another. Further, the processing done by the keyword expansion system 210 does not necessarily occur prior to the processing done by the keyword extraction system 212, and vice versa. The interactions between the keyword expansion system 210 and the keyword extraction system 212 may be more circular than the linear flow depicted.

According to some examples, the NLP system 208 also performs relevance matching to determine relevancy of the set of found keywords. The NLP system 208 uses one or more NLP models to determine how relevant each found keyword in comparison to the initial set of keywords 204. For example, the NLP system 208 may use one or more BERTs and/or lemmatization model to determine the quantifiable relevance score of each found keyword. According to some embodiments, the NLP system 208 may request user input on the relevance of each found keyword to improve processing (e.g., via a report 216 caused for display to a user).

The keyword processing system 122 includes a post-processing system 214 that provides one or more post-processing functions. The post-processing system 214 receives at least any found keywords from the NLP system 208, and, if applicable, any additional data about the found keywords, such as relevant text excerpts. According to some examples, the post-processing system 214 generates a report 216.

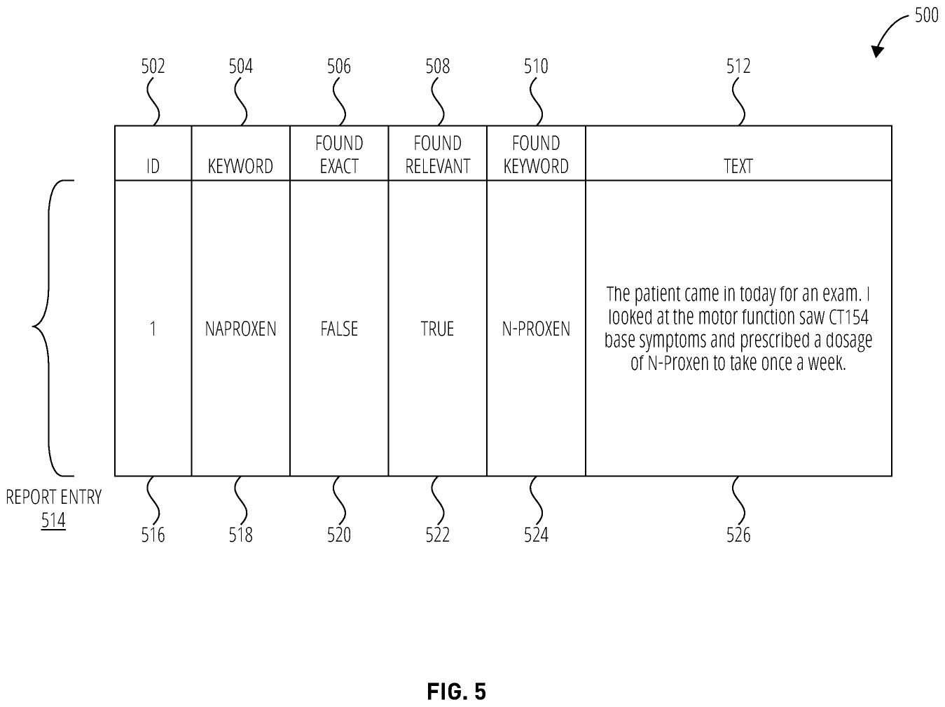

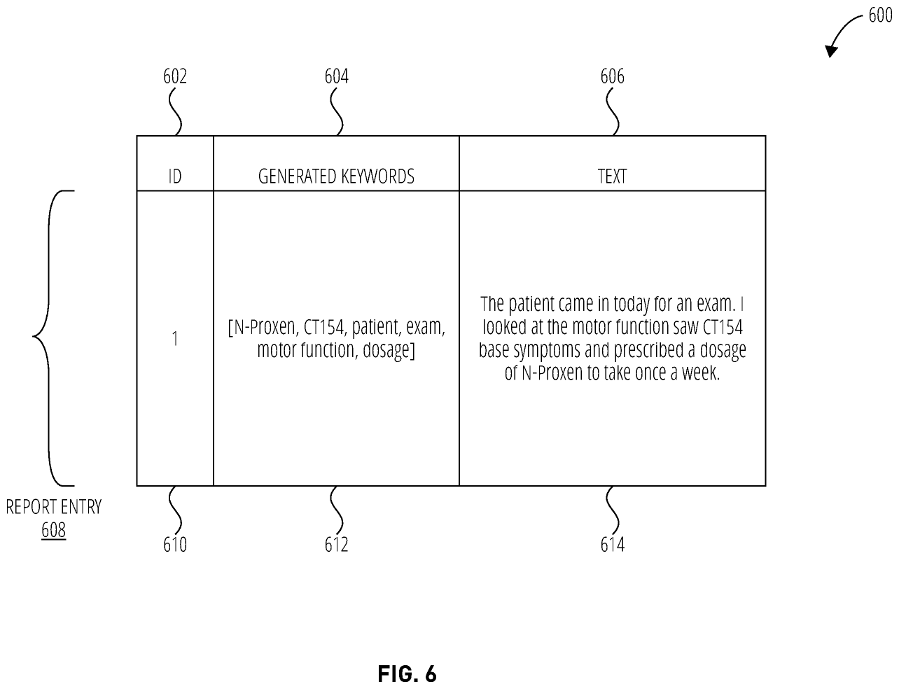

The report 216 includes at least any found keywords. According to some examples, the report 216 also includes at least one text excerpt from the target text 202, such that each found keyword in the set of found keywords is associated with a text excerpt from the target text 202. Example reports 216 are described in further detail in relation to FIG. 5 and FIG. 6.

The keyword processing system 122 may perform any of a plurality of actions with the report 216. For example, the keyword processing system 122 may transmit the report 216 to the database server 118 for storage in the database 120. Additionally, or alternatively, the keyword processing system 122 may transmit the report 216 to third-party servers 112 and/or any user system 104 via the network 102. According to some examples, the report 216 is transmitted with instructions, that when executed, cause for display of the report 216 at a user device, such as computer user device 114.

According to some examples, the functionality of the keyword processing system 122 is incorporated in the local application 106 of the user system 104. Accessing the keyword processing system 122 through the local application 106 can improve processing speeds by cutting down on network 102 requests. In the event the network 102 is interrupted or the network servers 116 are otherwise unavailable, the application 106 will be able to run the keyword processing system 122.

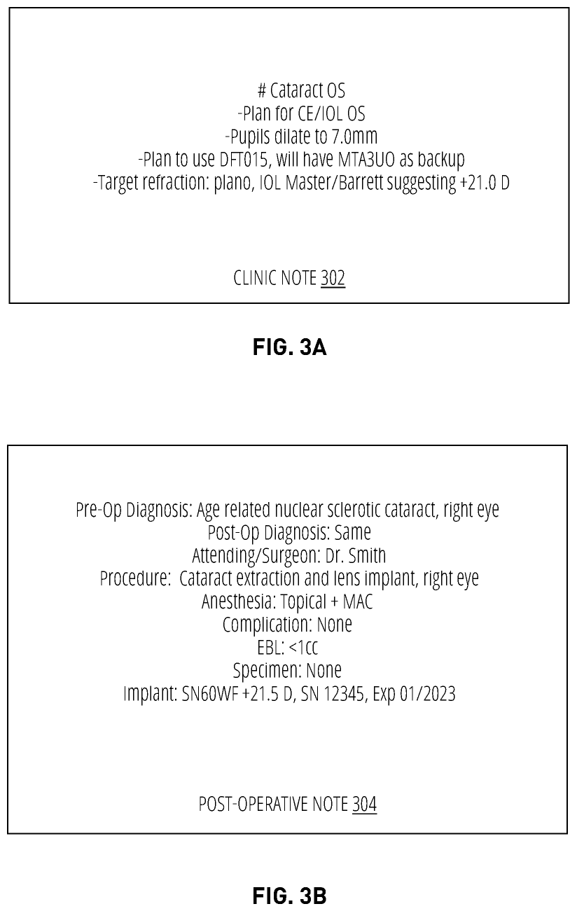

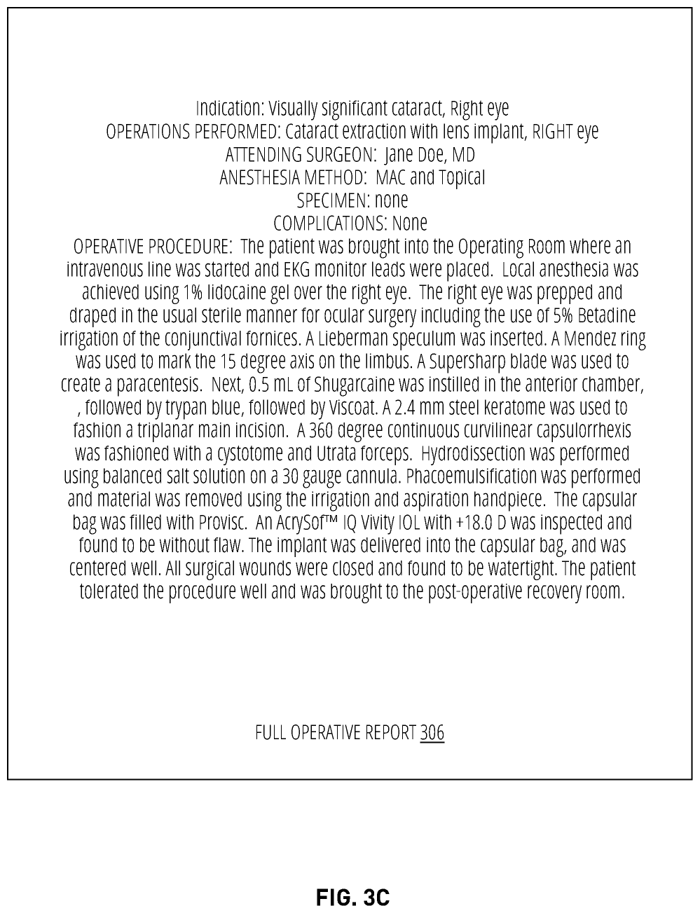

FIG. 3A illustrates an example target text, according to some examples. In this example, the target text is a clinic note 302. In general, a clinic note is a medical record about a patient, typically authored by a physician during an appointment with the patient. The keyword processing system 122 is configured to determine relevant keywords and associated relevant text from the clinic note 302.

The clinic note 302 references “IOL,” which is an acronym for intraocular lens. IOLs are artificial implants used to replace a patient's natural crystalline lens during cataract surgery. There are numerous brand and/or trade names associated with each IOL. The specific IOL used has clinical implications because each different IOL may have different complications and side effect profiles, depending on the design of the IOL and the material used to fabricate the IOL. Accordingly, accurate identification of an IOL is important as a variable for real-world research studies, clinical trials, as well as in ordinary clinical practice.

The way healthcare providers may document IOLs used can vary, as shown in FIG. 3A, FIG. 3B, and FIG. 3C. These variations can make reviewing medical records to identify which IOL a patient has quite time consuming and prone to human error. However, the keyword processing system 122 is configured to determine relevant information, such as the IOL used, quickly and without being prone to human error.

For example, the keyword processing system 122 identifies and extracts the keyword “DFT015,” which represents the model number of the primary IOL, and associated relevant text “plan to use DFT015.” According to some examples, the keyword processing system 122 also identifies and extracts the keyword “MTA3UO,” which represents the model number of the backup IOL, and associated relevant text “will have MTA3UO as a backup.” Additionally, the keyword processing system 122 may identify and extract the keyword “+21.0 D,” which represents the associated lens dioptric power of the primary IOL, and associated relevant text “IOL Master/Barrett suggesting +21.0 D.” According to some examples, the keyword processing system 122 identifies the keywords “DFT015” and “+21.0 D” are relevant to one another and may associate the two keywords in the report 216, for example.

FIG. 3B illustrates an example target text, according to some examples. In this example, the target text is a post-operative note 304. In general, a post-operative note is a medical record that is a short summary of a procedure (e.g., a surgical procedure). Typically, a post-operative note is drafted for informing medical team members of the nature of the procedure after the procedure has occurred but prior to a physician (e.g., the surgeon) drafting a full operative report. The procedure the post-operative note 304 is about is implantation of an IOL. The keyword processing system 122 is configured to process the post-operative note 304 and extract relevant keywords and text.

For example, the keyword processing systems 122 identifies and extract the keyword “right eye,” which represents the laterality of the procedure, and relevant text, “Procedure: Cataract extraction and lens implant, right eye.” Additionally, the keyword processing system 122 identifies and extracts the keyword “SN60WF,” which represents the model number of the IOL implanted in the patient, and associated relevant text “Implant: SN60WF +21.5 D, SN 12345, Exp 01/2023.” The keyword processing system 122 additionally identifies and extracts the keyword “+21.5 D,” which represents the dioptric power of the implanted IOL, and associated relevant text “Implant: SN60WF +21.5 D, SN 12345, Exp 01/2023.” Note the keywords “SN60WF” and “+21.5 D” are both associated with the same relevant text. According to some examples, the report 216 generated by the keyword processing system 122 relates these two keywords, presenting them together in the report 216 with the shared relevant text.

FIG. 3C illustrates an example target text, according to some examples. In this example, the target text 202 is a full operative report 306. In general, a full operative report is a detailed description of a procedure (e.g., surgical procedure), typically authored by a physician involved in the procedure (e.g., surgeon). The full operative report 306 provides more information about the IOL implantation procedure than a post-operative note. The keyword processing system 122 is configured to process the full operative report 306 and extract relevant keywords and text.

For example, the keyword processing system 122 determines the term “RIGHT eye” as a keyword, representing the laterality of the procedure, and associated relevant text, “OPERATIONS PERFORMED: Cataract extraction with lens implant, RIGHT eye.” Further, the keyword processing system 122 determines “AcrySof™ IQ Vivity IOL model with +18.0 D” is a keyword, representing the specific type of IOL lens implanted in the patient, along with relevant text, “An AcrySof™ IQ Vivity IOL model with +18.0 D was inspected and found to be without flaw.” Note this keyword is equivalent in meaning to its model number, “DFT315,” as identified as a keyword in the clinic note 302. In an example where the keyword processing system 122 processes both the clinic note 302 and the full operative report 306 of the same patient, the keyword processing system 122 is configured to properly categorize these keywords as equivalent. The keyword processing system 122 may further identify the keyword “15 degree axis,” which is specific to the type of IOL used, and relevant text “A Mendez ring was used to mark the 15 degree axis on the limbus.” For example, the keyword “15 degree axis,” can be used to further confirm the type of IOL implanted, as only certain IOL types require positioning at a specific orientation.

The example target texts provided in FIG. 3A, FIG. 3B, and FIG. 3C illustrate the heterogeneity of data and terminology in medical records, even within the specific discipline of IOL implantation. The keyword processing system 122 is able to parse through the heterogeneous terms and extract pertinent data, such as keywords and their associated relevant text. In research use, the keyword processing system 122 can save researchers weeks to months of research time by quickly and robustly parsing target texts.

Furthermore, in a clinical setting, such as a physician's office, the keyword processing system 122 can be used to increase cost-effectiveness of healthcare outcomes. For example, many health insurance companies provide merit-incentive payment systems (MIPS), which are payments that are based on the outcomes of healthcare. When submitting a procedure for reimbursement to a health insurance company, a procedure that meets the criteria for MIPS will receive an additional merit-based payment. Typically, MIPS have extremely specific criteria (e.g., 40-50 different measures) that must be documented when submitting for reimbursement. Conventionally, too many human-hours are required to review documentation and determine whether any given procedure outcome qualifies for MIPS. Accordingly, MIPS is not financially viable for many healthcare providers, especially smaller clinics, to pursue. However, the keyword processing system 122 facilitates extracting relevant criteria to meet MIPS for any given procedure, thereby making MIPS submission faster and more cost-effective. Receiving more merit-based payments further incentivizes better healthcare outcomes.

FIG. 4 illustrates several exemplary types of keyword expansions, according to some examples. The NLP system 208 in general, and the keyword expansion system 210 in particular, are configured to expand upon a given keyword using one or more NLP models (e.g., BERTs). For example, the NLP system 208 may expand upon the given keyword using one or more of the exemplary types of keyword expansions illustrated in FIG. 4 as well as any additional types of keyword expansion not pictured.

According to some examples, the NLP system 208 uses different words with the same meaning 402 to expand on any given keyword. For example, the keywords 404 “Cyclosporine,” “Ciclosporin,” “Neoral,” and “Sandimmune,” are all different words with the same meaning 402. That is, for a given keyword, “Cyclosporine,” the NLP system 208 expands the set of keywords to also include “Ciclosporin,” “Neoral,” and “Sandimmune,” because they are each a different word with the same meaning 402. The NLP system 208 may determine which keywords are different words with the same meaning 402 using one or more BERTs, using training data, using thesaurus data, using a large-language model (LLM) and/or using user input, among other means.

According to some examples, the NLP system 208 uses different grammar with the same meaning 406 to expand on any given keyword. For example, the keywords 408 “5 mg/kg of Neoral per day,” “5 mg/kg per diem of Neoral,” and “Neoral 5 mg/5 kg per day,” are all different grammar with the same meaning 406. That is, for a given keyword “5 mg/kg of Neoral per day,” the NLP system 208 expands the set of keywords to also include “5 mg/kg per diem of Neoral,” and “Neoral 5 mg/5 kg per day,” because they are each a different grammar with the same meaning 406. The NLP system 208 may determine which keywords have a different grammar with the same meaning 406 as a given keyword using one or more BERTs (e.g., a sentence-based BERT), using training data, using a large-language model (LLM) and/or using user input, among other means.

According to some examples, the NLP system 208 uses different expressions with the same meaning 410 to expand on any given keyword. For example, the keywords 412 “Non-smoker,” “Does not smoke,” “Does not drink or smoke,” and “Denies tobacco use,” are all different expressions with the same meaning 410 as related to tobacco use. That is, for a given keyword of “Non-smoker,” the NLP system 208 expands the set of keywords to also include “Does not smoke,” “Does not drink or smoke,” and “Denies tobacco use,” because they are each a different expression with the same meaning 410 as related to tobacco use. The NLP system 208 may determine which keywords have a different expression with the same meaning 410 as a given keyword using one or more BERTs (e.g., a sentence-based BERT), using training data, using a large-language model (LLM) and/or using user input, among other means.

According to some examples, the NLP system 208 uses the same word in different context 414 to expand on any given keyword. For example, the keywords 416 “Diagnosed with diabetes,” “Family history of diabetes,” and “No family history of diabetes,” all contain the same word in different context 414 (i.e., the word is ‘diabetes’). The NLP system 208 may determine which keywords have the same word in different context 414 as a given keyword using one or more BERTs (e.g., a sentence-based BERT), using training data, using a large-language model (LLM) and/or using user input, among other means.

According to some examples, the NLP system 208 uses more than one type of keyword expansion. Additionally, or alternatively, the NLP system 208 may recursively expand keywords. For example, given the keyword, “5 mg/kg of Neoral per day,” the NLP system 208 expands the set of keywords to include keywords with different grammar with the same meaning 406 (e.g., “5 mg/kg per diem of Neoral,” and “Neoral 5 mg/5 kg per day”). The NLP system 208 identifies “Neoral,” as an independent keyword, for example by identifying it as a medication name, and expands on the keyword to include different words with the same meaning 402 (e.g., “Ciclosporin,” “Neoral,” and “Sandimmune”). For completeness, the NLP system 208 recursively expands the keywords with different grammar with the same meaning 406 to include the different words with the same meaning 402. That is, “5 mg/kg of Ciclosporin per day,” “5 mg/kg per diem of Ciclosporin,” “Ciclosporin 5 mg/5 kg per day,” “5 mg/kg of Sandimmune per day,” “5 mg/kg per diem of Sandimmune,” “Sandimmune 5 mg/5 kg per day,” etc., are included in the expanded set of keywords.

In such cases, the NLP system 208 may perform multiple rounds of keyword expansion based on configurations of the keyword processing system 122. According to some examples, the stopping condition of the recursive expansion depends on the set of found keywords in the target text. For example, the keyword processing system 122 may be configured to continue to expand on keywords until a predetermined (e.g., threshold) number of keywords are found in the target text.

In another example, the keyword processing system 122 performs relevancy matching on the set of found keywords, for example through the post-processing system 214, and recursively generates more keywords in the expanded set of keywords until the relevancy matching exceeds a threshold. According to some examples, a user can define a threshold of embedded relevance a keyword must have to be added to the set of found keywords.

For example, the keywords “surgical” and “surgery” have an embedded relevance of 0.91, while the keywords “medicine” and “acetaminophen” have an embedded relevance of 0.65. Embedded relevance can be determined using one or more NLP models (e.g., BERTs) of the NLP system 208. The keyword processing system 122 determines additional keywords of relevance within the threshold. According to some such examples, the report 216 includes an indication of whether each found keyword is an exact match or a relevant match, such as the example report of FIG. 5. Additionally, or alternatively, the report 216 includes an indication of the embedded relevancy between the target keyword and found relevant keyword. For example, the target keyword is “surgical” and found relevant keyword is “surgery,” the embedded relevancy is indicated as 0.91.

FIG. 5 illustrates an example report 500, according to some examples. The report 500 is an example UI of a report 216 generated by the keyword processing system 122 from an associated target text, according to some examples. The report 500 is a table including column headers for ID 502, keyword 504, found exact 506, found relevant 508, found keyword 510, and text 512. The report 500 includes a report entry 514 of a found keyword as an illustrative example.

The ID 502 column contains an identification for the report entry 514. For example, the report entry 514 includes a sample ID 516 of “1.” According to other examples, the ID 502 may be a numeric or alphanumeric identifier of the report entry 514.

The keyword 504 column contains a keyword that was searched for. That is, in the example report 500, the keyword 504 column includes a keyword that was part of the initial set of keywords (e.g., provided by user input). For example, the report entry 514 includes a sample keyword 518 of “Naproxen.” According to other examples, the keyword 504 may be any keyword that is a word or phrase to be searched for in the associated target text. According to some examples, the keyword 504 column may include multiple entries.

The found exact 506 column contains an indication whether the keyword 504 was found in the target text. That is, in the example report 500, the found exact 506 column contains a binary indication of whether the exact keyword specified in the keyword 504 column was found in the associated target text. For example, the found exact 506 column includes a sample found exact 520 of “false,” indicating the sample keyword 518 “Naproxen” was not found in the target text. In an example wherein the sample keyword 518 was found in the target text, the found exact 506 column would contain the entry “true.”

The found relevant 508 column contains an indication whether a different keyword based on an expansion of the keyword 504 was found in the target text. That is, in the example report 500, the found relevant 508 column contains a binary indication of whether a related keyword (e.g., a keyword determined by the NLP system 208 based on the keyword 504) was found in the target text. For example, the found relevant 508 column includes a sample found relevant 522 of “true,” indicating a keyword related to the sample keyword 518 “Naproxen” was found in the target text. In an example wherein a keyword related to the sample keyword 518 was not found in the target text, the found relevant 508 column would contain the entry “false.”

According to other examples, the found exact 506 column and the found relevant 508 column may contain any other type of entry used to indicate a binary result, such as numbers (e.g., “0” and “1”), symbols (e.g., “X” and a check mark), other words (e.g., “not found” and “found”), and the like.

The found keyword 510 column specifies which keyword(s) were found in the target text. That is, if the keyword specified in the keyword 504 column were found, the found keyword 510 column would include the same sample keyword 518 entry. In the example report 500, as indicated by the sample found relevant 522, a keyword related to the sample keyword 518 was found. The found keyword 510 column includes a sample found keyword 524, “N-proxen.” Note the sample found keyword 524, “N-proxen,” and the sample keyword 518, “Naproxen,” are different words with the same meaning 402. In examples where more than one keyword related to the keyword 504 and/or text 512 are found, the found keyword 510 column includes multiple entries. For example, if sample found exact 520 were “true,” then the found keyword 510 column would include “N-proxen” and “Naproxen.”

The text 512 column includes one or more relevant text passages to the found keyword 510, as determined by the keyword processing system 122. In general, the text 512 column includes relevant text passages that include the found keyword 510. In the report 500, the relevant text associated with the sample found keyword 524 “N-proxen” is a sample text 526, “The patient came in today for an exam. I looked at the motor function saw CT154 base symptoms and prescribed a dosage of N-proxen to take once a week.” In examples where the sample found keyword 524 is found in more than one passage in the target text, the text 512 column may include multiple entries. In examples where the found keyword 510 column includes multiple entries, the text 512 column may include multiple entries.

A report 216 may contain fewer or additional rows and columns than the report 500. For example, if no keywords are found, the found keyword 510 column and the text 512 column may be omitted from the report 500. The report 500 may also be presented as part of a larger UI that is caused for display on a user device. The report 500 may be formatted differently than the table depicted for presentation on a user device. The report 500 may include additional functionality, such as links to view the associated target text in its entirety.

FIG. 6 illustrates an example report, according to some examples. The report 600 is an example UI of a report 216 generated by the keyword processing system 122 from an associated target text, according to some examples. The report 600 is a table including column headers for ID 602, generated keywords 604, and text 606. The report 600 includes a report entry 608 of a found keyword as an illustrative example. According to some examples, the report 600 is an example output in embodiments without an initial set of keywords.

The ID 602 column contains an identification for the report entry 608. For example, the report entry 608 includes a sample ID 610 of “1.” According to other examples, the ID 602 may be a numeric or alphanumeric identifier of the report entry 608.

The generated keywords 604 column contains a keyword that were generated by the NLP system 208 based on the target text. That is, the NLP system 208 processes the target text to extract relevant text and expand keywords in conjunction with one another. The NLP system 208 may be configured to first locate one specific type of keyword, such as medication names, and expand the set of keywords based on found medication names and their associated relevant text. For example, the report entry 608 includes a list of sample generated keywords 612, “[N-Proxen, CT154, patient, exam, motor function, dosage],” where “N-proxen” is a keyword representing a medication name and the remaining keywords are extracted from the same relevant text in the text 606 column. In some examples, the generated keywords 604 list may include one or zero entries.

The text 606 column includes a relevant text passage to the generated keywords 604. That is, in the example report 600, each list of entries in the generated keywords 604 column is associated with an entry in the text 606 column. For example, associated with the list of sample generated keywords 612 is a sample text 614, “The patient came in today for an exam. I looked at the motor function saw CT154 base symptoms and prescribed a dosage of N-Proxen to take once a week.” According to other embodiments, the text 606 column may include multiple entries.

A report 216 may contain fewer or additional rows and columns than the report 600. For example, if no keywords are found, the report 600 may present differently. The report 600 may also be presented as part of a larger UI that is caused for display on a user device. The report 600 may be formatted differently than the table depicted for presentation on a user device. The report 600 may include additional functionality, such as links to view the associated target text in its entirety.

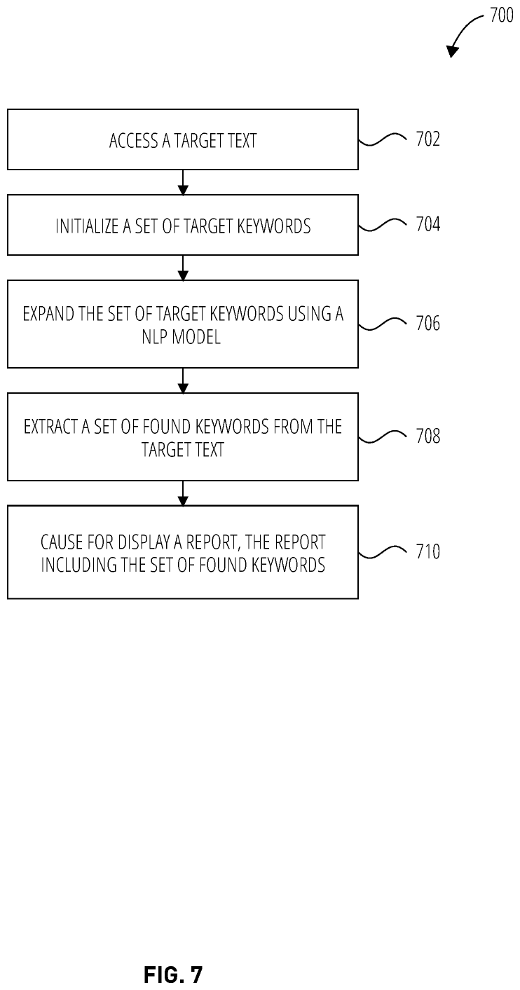

FIG. 7 is a flowchart of a method 700 of processing a target text, according to some examples. The method 700 can be performed by processing logic that can include hardware (e.g., a processing device, circuitry, dedicated logic, programmable logic, microcode, hardware of a device, an integrated circuit, etc.), software (e.g., instructions run or executed on a processing device), or a combination thereof. In some embodiments, the method 700 is performed by functional components of the system 100. While the operations below are described as being performed by a processing device, it shall be appreciated that the operations of the method 700 may not necessarily be performed by the same processing device. Accordingly, any one or more of the operations of the method 700 can be performed by any one or more of the user system 104, the network server system 110, or any combination thereof. The processing device may, for example, be the keyword processing system 122.

Although shown in a particular sequence or order, unless otherwise specified, the order of the processes can be modified. Thus, the illustrated embodiments should be understood only as examples, and the illustrated processes can be performed in a different order, and some processes can be performed in parallel. Additionally, one or more processes can be omitted in various embodiments. Thus, not all processes are required in every embodiment. Other process flows are possible.

At block 702, the processing device accesses a target text. According to some examples, the processing device accesses more than one target text, which may be combined into a singular target text for processing. The target text may be accessed, or otherwise received from, a database, such as database 120. Each target text includes text data, generally unstructured text data. According to some embodiments, the target text is part of a medical record of a patient.

At block 704, the processing device initializes a set of target keywords. The keywords are words or phrases to be searched for in the target text. According to some examples, the processing device receives an initial set of keywords from a user input, for example, through a user system 104. According to some examples, the processing device receives, or otherwise accesses, an initial set of keywords from a database, such as database 120. According to some examples, the processing device does not receive an initial set of keywords and initializes an empty set of keywords. According to some examples, the processing device does not receive an initial set of keywords and initializes set of keywords that includes potentially relevant medication names.

At block 706, the processing device expands the set of target keywords using an NLP model. According to some examples, the processing device uses one or more NLP models to expand the set of target keywords. According to some examples, the processing device expands the set of target keywords based on the initial set of keywords. For example, the initial set of target keywords may include a name of a medication. The expanded set of target keywords includes one or more alternative names for the medication.

According to some examples, to expand the set of target keywords, the processing device determines a first set of additional target keywords that includes keywords that are synonyms of the keyword (e.g., different words with the same meaning 402). According to some examples, the processing device determines a second set of additional target keywords that includes keywords with different grammar than the keyword and synonymous meaning to the keyword (e.g., different grammar with the same meaning 406). According to some examples, the processing device determines a third set of additional target keywords that includes keywords with different expressions than the keywords and synonymous meaning to the keyword (e.g., different expression with the same meaning 410). According to some examples, the processing device determines a fourth set of additional target keywords that includes keywords that comprise the keyword in one or more different contexts (e.g., same word in different context 414). The expanded set of keywords includes any combination of the initial set of keywords, the first set of additional target keywords, the second set of additional target keywords, the third set of additional target keywords, and the fourth set of additional target keywords.

At block 708, the processing device extracts a set of found keywords from the target text. According to some examples, the set of found keywords are extracting using one or more NLP models, which may be the same NLP model(s) used to expand the set of target keywords. The processing device extracts the set of found keywords from the target text based on the expanded set of target keywords. According to some examples, the processing device extracts additional data about the set of found keywords, such as relevant text associated with the found keywords.

According to some examples, the processing device may perform refined relevancy matching on the set of found keywords using the one or more NLP models. The one or more NLP models may use the relevancy matching to further expand the expanded set of target keywords. The relevancy matching may also be used to further refine the set of found keywords.

At block 710, the processing device causes for display a report, the report including at least the set of found keywords. According to some examples, the report includes at least one text excerpt from the target text. Further, according to some examples, each found keyword in the set of found keywords is associated with at least one text excerpt from the target text. The report may contain additional information about the found keywords and/or the target text. The processing device causes the report for display, for example on a display of a user system 104. The processing device may transmit instructions for causing the display over a network, such as network 102.

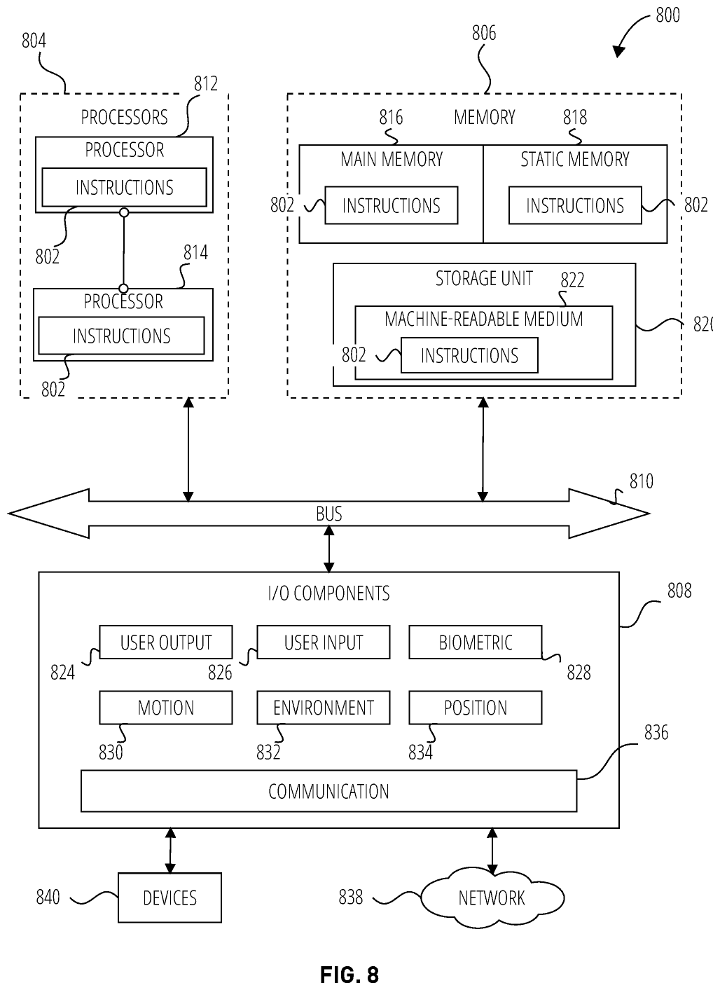

FIG. 8 is a diagrammatic representation of the machine 800 within which instructions 802 (e.g., software, a program, an application, an applet, an app, or other executable code) for causing the machine 800 to perform any one or more of the methodologies discussed herein may be executed. For example, the instructions 802 may cause the machine 800 to execute any one or more of the methods described herein. The instructions 802 transform the general, non-programmed machine 800 into a particular machine 800 programmed to carry out the described and illustrated functions in the manner described. The machine 800 may operate as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 802, sequentially or otherwise, that specify actions to be taken by the machine 800. Further, while a single machine 800 is illustrated, the term “machine” shall also be taken to include a collection of machines that individually or jointly execute the instructions 802 to perform any one or more of the methodologies discussed herein. The machine 800, for example, may comprise the user system 104 or any one of multiple server devices forming part of the network server system 110. In some examples, the machine 800 may also comprise both client and server systems, with certain operations of a particular method or algorithm being performed on the server-side and with certain operations of the particular method or algorithm being performed on the client-side.

The machine 800 may include processors 804, memory 806, and input/output I/O components 808, which may be configured to communicate with each other via a bus 810. In an example, the processors 804 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 812 and a processor 814 that execute the instructions 802. The term “processor” is intended to include multi-core processors that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiples cores, or any combination thereof.

The memory 806 includes a main memory 816, a static memory 818, and a storage unit 820, both accessible to the processors 804 via the bus 810. The main memory 806, the static main memory 816, and storage unit 820 store the instructions 802 embodying any one or more of the methodologies or functions described herein. The instructions 802 may also reside, completely or partially, within the main memory 816, within the static memory 818, within machine-readable medium 822 within the storage unit 820, within at least one of the processors 804 (e.g., within the processor's cache memory), or any suitable combination thereof, during execution thereof by the machine 800.

The I/O components 808 may include a wide variety of components to receive input, provide output, produce output, transmit information, exchange information, capture measurements, and so on. The specific I/O components 808 that are included in a particular machine will depend on the type of machine. For example, portable machines such as mobile phones may include a touch input device or other such input mechanisms, while a headless server machine will likely not include such a touch input device. It will be appreciated that the I/O components 808 may include many other components that are not shown in FIG. 8. In various examples, the I/O components 808 may include user output components 824 and user input components 826. The user output components 824 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 826 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 808 may include biometric components 828, motion components 830, environmental components 832, or position components 834, among a wide array of other components. For example, the biometric components 828 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 830 include acceleration sensor components (e.g., accelerometer), gravitation sensor components, rotation sensor components (e.g., gyroscope).

The environmental components 832 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

With respect to cameras, the user system 104 may have a camera system comprising, for example, front cameras on a front surface of the user system 104 and rear cameras on a rear surface of the user system 104. In addition to front and rear cameras, the user system 104 may also include a 360° camera for capturing 360° photographs and videos. Further, the camera system of the user system 104 may include dual rear cameras (e.g., a primary camera as well as a depth-sensing camera), or even triple, quad or penta rear camera configurations on the front and rear sides of the user system 104. These multiple cameras systems may include a wide camera, an ultra-wide camera, a telephoto camera, a macro camera, and a depth sensor, for example.

The position components 834 include location sensor components (e.g., a GPS receiver components), altitude sensor components (e.g., altimeters or barometers that detect air pressure from which altitude may be derived), orientation sensor components (e.g., magnetometers), and the like.

Communication may be implemented using a wide variety of technologies. The I/O components 808 further include communication components 836 operable to couple the machine 800 to a network 838 or devices 840 via respective coupling or connections. For example, the communication components 836 may include a network interface component or another suitable device to interface with the network 838. In further examples, the communication components 836 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 840 may be another machine or any of a wide variety of peripheral devices (e.g., a peripheral device coupled via a USB).

Moreover, the communication components 836 may detect identifiers or include components operable to detect identifiers. For example, the communication components 836 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 836, such as location via Internet Protocol (IP) geolocation, location via Wi-Fi® signal triangulation, location via detecting an NFC beacon signal that may indicate a particular location, and so forth.

The various memories (e.g., main memory 816, static memory 818, and memory of the processors 804) and storage unit 820 may store one or more sets of instructions and data structures (e.g., software) embodying or used by any one or more of the methodologies or functions described herein. These instructions (e.g., the instructions 802), when executed by processors 804, cause various operations to implement the disclosed examples.

The instructions 802 may be transmitted or received over the network 8388, using a transmission medium, via a network interface device (e.g., a network interface component included in the communication components 836) and using any one of several well-known transfer protocols (e.g., hypertext transfer protocol (HTTP)). Similarly, the instructions 802 may be transmitted or received using a transmission medium via a coupling (e.g., a peer-to-peer coupling) to the devices 840.

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

What is claimed is:

1. A system comprising:
one or more processors of a machine; and
a memory storing instructions that, when executed by the one or more processors, cause the machine to perform operations comprising:
accessing a set of medical records, the set of medical records including at least a target text;
initializing a set of target keywords;
expanding the set of target keywords using one or more natural language processing (NLP) models based on the initialized set of target keywords;
extracting a set of found keywords by using the one or more NLP models to process the target text with the expanded set of target keywords;
causing for display a report, the report including the set of found keywords and at least one text excerpt from the target text, wherein each found keyword in the set of found keywords is associated with a text excerpt from the target text.

2. The system of claim 1, wherein the target text is unstructured data.

3. The system of claim 1, wherein initializing the set of target keywords comprises:
receiving a set of input words from a user;
initializing the set of target keywords; the set of target keywords including at least the set of input words.

4. The system of claim 1, wherein initializing the set of target keywords comprises:
initializing the set of target keywords as an empty set.

5. The system of claim 4, wherein expanding the set of target keywords using the NLP model based on the set of initial target keywords further comprises:
extracting a set of generalized keywords from the target text.

6. The system of claim 1, wherein the set of target keywords includes a medication.

7. The system of claim 6, wherein the expanded set of target keywords includes one or more alternative names for the medication.

8. The system of claim 1, the operations further comprising:
performing refined relevancy matching on the set of found keywords using the NLP model based on the found keywords.

9. The system of claim 1, wherein expanding the set of target keywords the NLP model based on the set of initial target keywords further comprises, for a keyword:
determining a first set of additional target keywords, the first set of additional target keywords including keywords that are synonyms of the keyword;
determining a second set of additional target keywords, the second set of additional target keywords including keywords with different grammar than the keyword and synonymous meaning to the keyword;
determining a third set of additional target keywords, the third set of additional target keywords including keywords with different expressions than the keywords and synonymous meaning to the keyword; and
wherein the expanded set of target keywords comprises the initialized set of target keywords, the first set of additional target keywords, the second set of additional target keywords, and the third set of additional target keywords.

10. The system of claim 9, wherein expanding the set of target keywords the NLP model based on the set of initial target keywords further comprises, for the keyword:
determining a fourth set of additional target keywords, the fourth set of additional target keywords including keywords that comprise the keyword in one or more different contexts; and
wherein the expanded set of target keywords further comprises the fourth set of additional target keywords.

11. A method comprising:
accessing a set of medical records, the set of medical records including at least a target text;
initializing a set of target keywords;
expanding the set of target keywords using one or more natural language processing (NLP) models based on the initialized set of target keywords;
extracting a set of found keywords by using the one or more NLP model to process the target text with the expanded set of target key words;
causing for display a report, the report including the set of found keywords and at least one text excerpt from the target text, wherein each found keyword in the set of found keywords is associated with a text excerpt from the target text.

12. The method of claim 11, wherein initializing the set of target keywords comprises:
receiving a set of input words from a user;
initializing the set of target keywords; the set of target keywords including at least the set of input words.

13. The method of claim 11, wherein initializing the set of target keywords comprises:
initializing the set of target keywords as an empty set.

14. The method of claim 13, wherein expanding the set of target keywords using the NLP model based on the set of initial target keywords further comprises:
extracting a set of generalized keywords from the target text.

15. The method of claim 11, wherein the set of target keywords includes a medication.

16. The method of claim 15, wherein the expanded set of target keywords includes one or more alternative names for the medication.

17. The method of claim 11, further comprising:
performing refined relevancy matching on the set of found keywords using the NLP model based on the found keywords.

18. The method of claim 11, wherein expanding the set of target keywords the NLP model based on the set of initial target keywords further comprises, for a keyword:
determining a first set of additional target keywords, the first set of additional target keywords including keywords that are synonyms of the keyword;
determining a second set of additional target keywords, the second set of additional target keywords including keywords with different grammar than the keyword and synonymous meaning to the keyword;
determining a third set of additional target keywords, the third set of additional target keywords including keywords with different expressions than the keywords and synonymous meaning to the keyword; and
wherein the expanded set of target keywords comprises the initialized set of target keywords, the first set of additional target keywords, the second set of additional target keywords, and the third set of additional target keywords.

19. The method of claim 18, wherein expanding the set of target keywords the NLP model based on the set of initial target keywords further comprises, for the keyword:
determining a fourth set of additional target keywords, the fourth set of additional target keywords including keywords that comprise the keyword in one or more different contexts; and
wherein the expanded set of target keywords further comprises the fourth set of additional target keywords.

20. A non-transitory computer-readable storage medium, the computer-readable storage medium including instructions that when executed by a computer, cause the computer to:
access a set of medical records, the set of medical records including at least a target text;
initialize a set of target keywords;
expand the set of target keywords using one or more natural language processing (NLP) models based on the initialized set of target keywords;
extract a set of found keywords by using the one or more NLP models to process the target text with the expanded set of target keywords;
cause for display a report, the report including the set of found keywords and at least one text excerpt from the target text, wherein each found keyword in the set of found keywords is associated with a text excerpt from the target text.
